(19) United States
(12) Patent Application Publication
(10) Pub. No.: US 2007/0207306 A1
(43) Pub. Date: Sep. 6, 2007

(54) EXPANDING TAPE BARRIER

(75) Inventors: Scott Broughton, Hollidaysburg, PA (US); R. Douglas Evans, Everett, PA (US); Timothy Ray McMillen, Tyrone, PA (US)

Correspondence Address: PIETRAGALLO, BOSICK & GORDON LLP, ONE OXFORD CENTRE, 38TH FLOOR, 301 GRANT STREET, PITTSBURGH, PA, 15219-6404, US

(73) Assignee: New Pig Corporation, Tipton, PA (US)

(21) Appl. No.: 11/681,855

(22) Filed: Mar. 5, 2007

Related U.S. Application Data
(60) Provisional application No. 60/779,350, filed on Mar. 3, 2006.

(51) Int. Cl. B32B 3/26; B32B 7/12; A61M 1/00; B32B 5/24

(52) U.S. Cl. 428/304.4; 428/343; 442/370; 604/317

(57) ABSTRACT

An expandable liquid barrier having an expandable layer attached to an adhesive layer is disclosed. The barrier provides constant containment without restricting access or creating a hazard in an area. The barrier is generally low profile like a length of tape until contacted by a liquid. Once in contact with a liquid the barrier expands and provides containment of the liquid. One means for expanding the barrier in the presence of liquid is to provide an expandable fill, such as super absorbent polymer, between the layers. Other means for releasing expanding the barrier include using a water-soluble adhesive to hold a resilient expanding layer and providing contracting section which increases the profile of the barrier. The barrier may be used in combination with a switch to enable reaction to the spill.

### CROSS REFERENCE TO RELATED APPLICATION

This application claims the benefit of U.S. Provisional Patent Application Ser. No. 60/779,350, filed Mar. 3, 2006, which is herein incorporated by reference.

### FIELD OF THE INVENTION

The present invention relates to containment and barriers for liquid spills.

### BACKGROUND INFORMATION

Curbs, barriers and berms often serve as containment for spilled liquids. These structures are often arranged to contain a liquid within the barrier or divert a liquid away to a more desirable location. Often the barriers are temporarily installed and removed when no longer needed. However, permanent structures are sometimes used where a recurring need exists.

Temporary barriers, such as berms, dykes or absorbent socks, can contain a spill or leak but must be installed to do so. Often it is the case that the leak or spill has occurred uncontained for some time before the temporary barriers are installed. This creates a much larger spill than would have occurred with a permanently installed barrier. Permanently installed barriers adequately provide constant containment. However, the permanent barriers also constantly project upward from the floor. This arrangement is usually undesirable because these structures create a trip hazard. Furthermore, the structures often create an impediment to wheeled traffic.

The present invention has been developed in view of the foregoing.

### SUMMARY OF THE INVENTION

The present invention provides an expandable liquid barrier having an expandable layer attached to an adhesive layer. The barrier provides constant containment without restricting access or creating a hazard in an area. The barrier is generally low profile, like a length of tape, until contacted by a liquid. Once in contact with a liquid the barrier expands and provides containment of the liquid. One means for expanding the barrier in the presence of liquid is to provide an expandable fill, such as super absorbent polymer, between the layers. Other means for releasing expanding the barrier include using a water-soluble adhesive to hold a resilient expanding layer and providing contracting section which increases the profile of the barrier. The barrier may be used in combination with a switch to enable reaction to the spill.

One aspect of the present invention provides an expandable liquid barrier comprising an adhesive layer attached to a base surface, an expanding layer attached to the adhesive layer and an expandable fill disposed between the adhesive layer and the expanding layer, wherein liquid contact with the expandable fill causes the expandable fill to swell.

Another aspect of the present invention provides an expandable liquid barrier comprising an adhesive layer attached to a base surface, an expanding layer attached to the adhesive layer comprising a resilient material and soluble adhesive, the expanding layer folded upon itself and restrained in a low profile position by the water-soluble adhesive, whereby liquid contact with the barrier causes the soluble adhesive to dissolve releasing the resilient expanding layer.

Another aspect of the present invention provides an expandable liquid barrier comprising an adhesive layer attached to a base surface, a contracting section connected to the adhesive layer and adjacent the base surface, an expanding layer attached to the contracting section, whereby liquid contact with the contracting section causes the contracting section to shrink which bends and increases the profile of the expanding layer.

Another aspect of the present invention provides an expandable liquid barrier comprising an adhesive layer attached to a base surface, an expanding layer attached to the adhesive layer and means for expanding and heightening the barrier when the barrier is contacted by a liquid.

Another aspect of the present invention provides a method for containing a leaking or spilled liquid comprising the steps of providing an expandable, low profile barrier which expands upon contact with the spilled or leaking liquid and affixing the expandable, low profile barrier to a base surface.

These and other aspects will become more apparent from the following description.

### BRIEF DESCRIPTION OF THE DRAWINGS

FIGS. 1A and 1B illustrate a perspective view of an expanding liquid barrier according to one embodiment of the present invention showing the low profile configuration of the barrier when liquid is not present and the expanded configuration when liquid contacts the barrier.

FIGS. 2A and 2B illustrate a cross-section of an expanding liquid barrier according to one embodiment of the present invention showing the adhesive layer, expanding layer and expanding fill.

FIG. 3 illustrates a top view of an expanding liquid barrier according to one embodiment of the present invention showing the front and rear longitudinal edges and the permeable and impermeable sections of the expanding layer wherein apertures are provided to create permeability.

FIG. 4 illustrate a top view of an expanding liquid barrier according to another embodiment of the present invention showing the front and rear longitudinal edges and the permeable and impermeable sections of the expanding layer wherein the permeable section liquid pervious without apertures.

FIG. 5A illustrates a top view of an expanding liquid barrier according to one embodiment of the present wherein the expanding layer is affixed to the adhesive layer at the rear longitudinal edge and the barrier is open at the front longitudinal edge.

FIG. 5B illustrates a front view of the barrier shown in FIG. 5A further showing the expandable fill and flexible members.

FIG. 5C illustrates a front view of the barrier shown in FIG. 5B showing the barrier's configuration when liquid is present.

FIG. 5D illustrates a side view of the barrier shown in FIG. 5C.

FIG. 6A illustrates illustrate a cross-section of an expanding liquid barrier according to one embodiment of the present invention having a resilient expanding layer held in a low profile position by water soluble absorbent.

FIG. 6B illustrates the barrier of 6A when liquid is present.

FIGS. 7A and 7B illustrate a cross-section of an expanding liquid barrier having a contracting section which increases the profile of the expanding section when liquid is present according to one embodiment of the present invention.

FIGS. 8A and 8B illustrates a cross-section of an expanding liquid barrier used in combination with a switch according to one embodiment of the present invention.

FIG. 9 illustrates how the expandable liquid barrier may serve to divert a liquid to a receptacle or drain.

### DETAILED DESCRIPTION

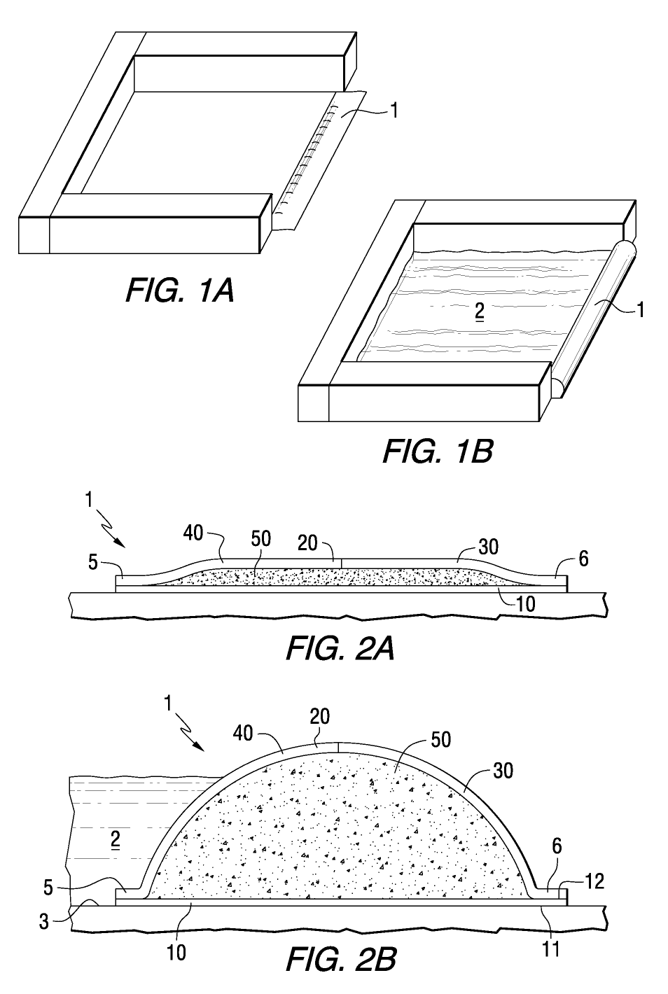

Referring now to FIG. 1A, an expanding liquid barrier 1 is shown according to one embodiment of the present invention. The expanding liquid barrier 1 is substantially flat and low profile when no liquid is present. The barrier 1 preferably has tape-like or narrow sheet-like geometry. When liquid 2 contacts the barrier 1, the barrier 1 heightens to create a barrier 1 or dyke as shown in FIG. 1B.

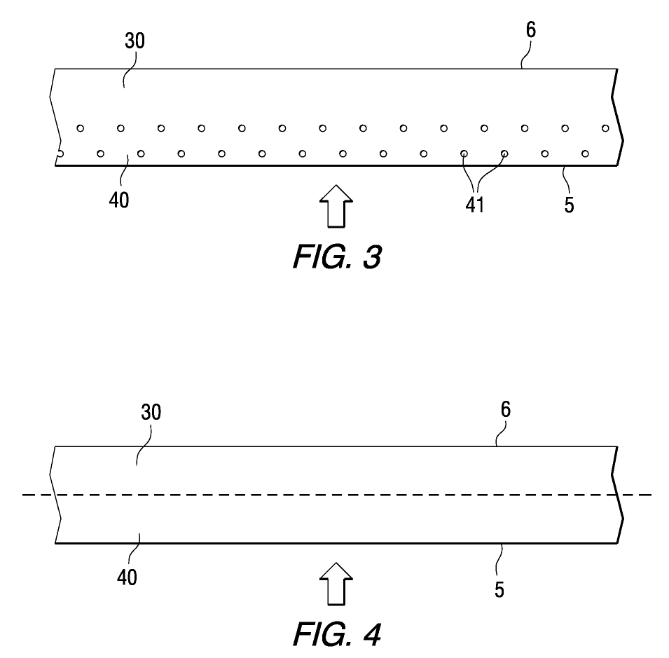

According to one embodiment of the present invention shown if FIGS. 2A and 2B, the barrier 1 may include an adhesive layer 10 which holds the barrier to a base surface 3. The base surface 3 may be any surface which may benefit from a barrier of this type, for example, a floor, walkway, entranceway, lab table, equipment platform or other surface. In one embodiment the adhesive layer 10 is a one-sided tape. The adhesive layer 10 has a bottom face 11 and a top face 12. The bottom face 11 is adhered to the base surface 3. The bottom face 11 of adhesive layer 10 prevents horizontal and vertical movement of the barrier 1. The bottom face 11 also creates a seal preventing liquid from passing under the barrier 1. The top face 12 is opposite the bottom face on the adhesive layer and is connected to an expanding layer 20. The expanding layer 20 is affixed to the top surface 12 of the adhesive layer 10 along a longitudinal edge of the barrier at a front end 5 and rear end 6. The expanding layer 20 may be affixed to the adhesive layer 10 by adhesive or by mechanical seal. The expanding layer 20 may be made of two or more sections, a liquid permeable section 40 and liquid impermeable section 30. The impermeable layer 30 may be made from any liquid impervious material, for example, vinyl, rubber, Acrylonitrile butadiene styrene (ABS), materials found under the tradename Sanoprene, polyethylene and polypropylene. The permeable section 40 may be made of perforated sheet and solid materials and woven and non-woven materials. The permeable layer 40 may be made from a material which allows fluid to pass through like a woven material or the liquid permeable layer 40 may be what is considered an impermeable material with various holes throughout the segment to allow liquid to pass as seen in FIG. 3.

Again referring to FIGS. 2A and 2B, an expandable fill 50 may be disposed between the adhesive layer 10 and the expanding layer 20. The expandable fill 50 is capable of increasing in size or swelling when it comes in contact with a liquid. As the expandable fill 50 swells the expanding layer 20 fills and increases in height. The expandable fill 50 may also absorb some amount of liquid. In one embodiment the expandable fill is a super absorbent polymer. In another embodiment the expandable fill 50 may comprise compressed sponges, foam or any other compressible absorbent. For example, Isocyanate-Based foams, Pyranyl foams, Syntactic foams, and/or Elastomeric foams may be used. Certain absorbents may be compressed through the use of a water-soluble binder, i.e. the absorbent may be wetted with the binder compressed and dried into a thin configuration.

Referring now to FIG. 3, a section of barrier 1 is shown. The arrow indicates the direction from which liquid would approach the barrier 1. In a preferred orientation, the permeable section 40 is positioned nearest the approach of the fluid. In this embodiment, permeable section 40 is typically made from the same material as impermeable section 30. The permeable section 40 allows fluid to pass by way of apertures 41.

Referring now to FIG. 4, the same preferred arrangement is shown with the permeable section nearest the approach of fluid. However, in this embodiment, the permeable section 40 and impermeable section 30 comprise different materials. The impermeable section 30 comprising a material impervious to the flow of liquid while the permeable section 40 utilizes and inherently permeable material to allow liquid to pass. The impermeable section 30 and permeable section 40 are joined by any common means.

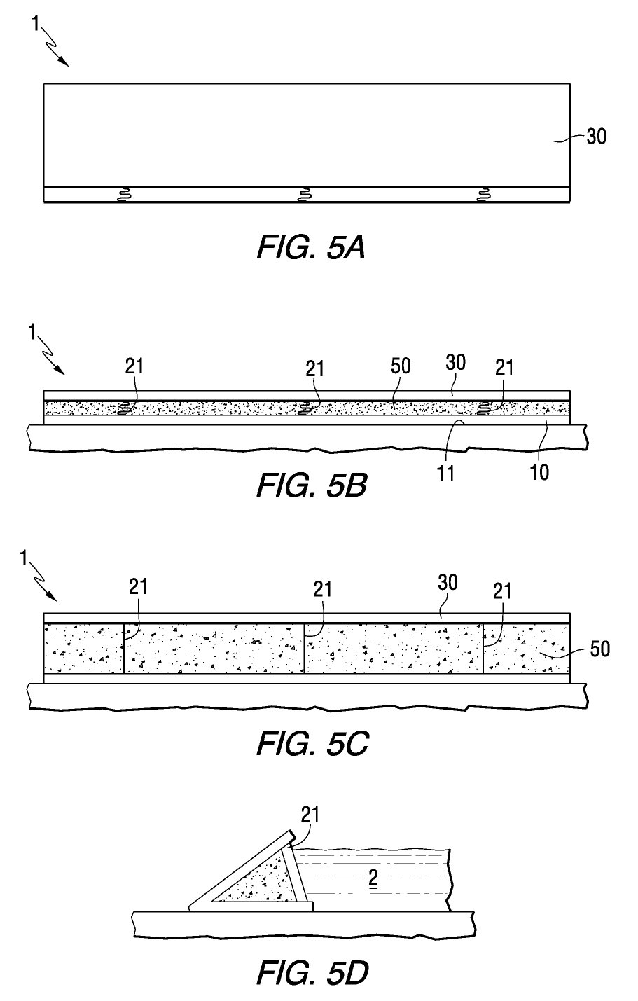

In the embodiment shown in FIGS. 5A-5D, the barrier 1 includes an adhesive layer 10 affixed at one end to an expanding layer 20. The expanding layer 20 includes only an impermeable section 30. The expanding layer 20 and adhesive layer 10 open at one end but are connected by flexible members 21. The flexible members 21 allow the expanding layer to open partially but not fully. For example, the flexible members 21 may restrict opening of the expanding layer 20 to less than 90°. As seen in FIG. 5B expandable fill 50 is disposed between the expanding layer 20 and adhesive layer 10. The expandable fill 50 swells when in contact with a liquid causing the expanding layer 20 to lift at its open end. The expanding layer 20 raises until the flexible members 21 are fully extended, as shown in FIG. 5C and FIG. 5D. Flexible members 21 may be made of any suitable material, which is flexible yet, substantially inelastic.

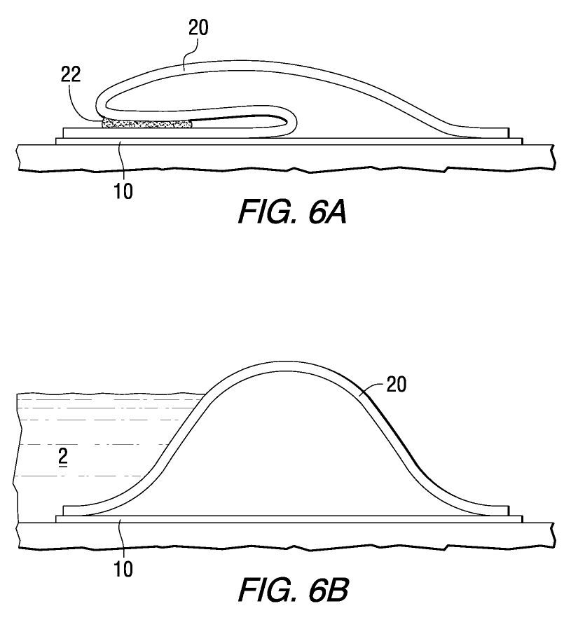

Another embodiment of an expanding liquid barrier according to the present invention is shown in FIGS. 6A and 6B. In this embodiment the barrier 1 has an adhesive layer 10 affixed to the floor and an expanding layer 20. The expanding layer 20 is made of an impermeable and highly resilient material, for example, rubber, ABS, materials found under the tradename Sanoprene, polyethylene, and polypropylene. As seen in FIG. 6A, the expandable layer 20 is held in a generally flat and low profile configuration by being compressed and held together with a water-soluble adhesive 22. When a water based liquid 2 comes into contact with the barrier 1 the water soluble adhesive 22 dissolves and releases the expanding layer so that the liquid may be contained as seen in FIG. 6B. The water soluble adhesive 22 may be, for example, cellulose ether, polyvinylpyrrolidone, or polyvinyl alcohol. It is also contemplated that the soluble adhesive 22 may be oil based where the barrier 1 is used to contain oil based liquids. Suitable oil soluble adhesives include, for example, Ethoxylated nonylphenol and Transitions metal alkyl sulfonates.

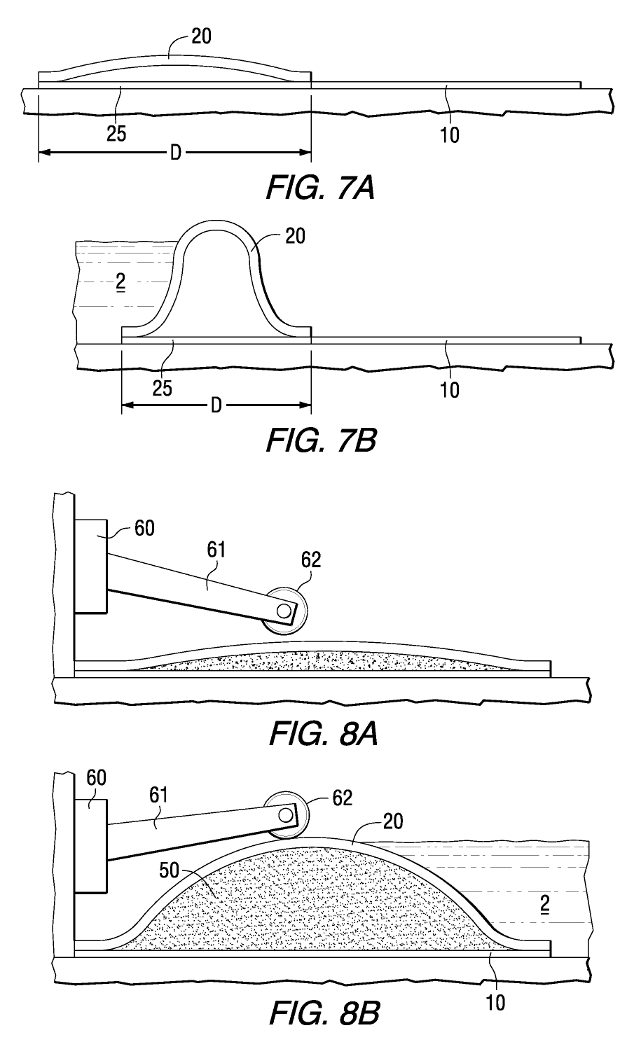

Referring now to FIGS. 7A and 7B, an expanding liquid barrier 1 according to one embodiment of the present invention is shown. In this embodiment, the barrier 1 has an adhesive layer 10 affixed to the floor. Adjacent the adhesive layer 10 is a contracting portion 25 also adjacent the floor. The expanding layer 20 is affixed at its ends to the contracting portion 25 as shown in FIG. 7A. In one embodiment the expanding layer 20 may have a front end attached to the contract section 25 and a rear end attached to the adhesive layer 10. The expanding layer 20 may be wholly made of liquid impermeable material or may have a portion which is permeable nearest the approach of the liquid 2 to be contained. When liquid 2 comes into contact with the contracting portion 25, the contracting portion 25 shrinks along dimension D is shown in FIGS. 7A and 7B. As the contracting portion 25 decreases along dimension D, the expanding layer 20 raises to contain the liquid 2 as shown in FIG. 7B.

Referring now to FIGS. 8A and 8B, a switch 60 may be incorporated with the barrier 1. The switch may be mounted so that the switch 60 is in one position when no liquid 2 is present and the barrier 1 is in a substantially flat arrangement. When liquid 2 is present the barrier 1 expands and activates the switch 60 as shown in FIG. 8B. The expanding barrier 1 and associated activation of switch 60 may send a signal to an alarm such as an audible alarm, beacon, strobe light or other indicator to alert personnel that a leak has been detected. It is also contemplated that the switch 60 could be tied into other equipment such as automatic valves, pumps, etc to stop the flow of liquid. This communication may be direct or through a computer or PLC. While the switch 60 shown in FIGS. 8A and 8B is a standard limit switch affixed with a metal lever 61 and wheel 62, it is contemplated that the switch 60 may be of any type suitable for this application. For example, the switch 60 could be a plunger type, proximity switch or other suitable switch.

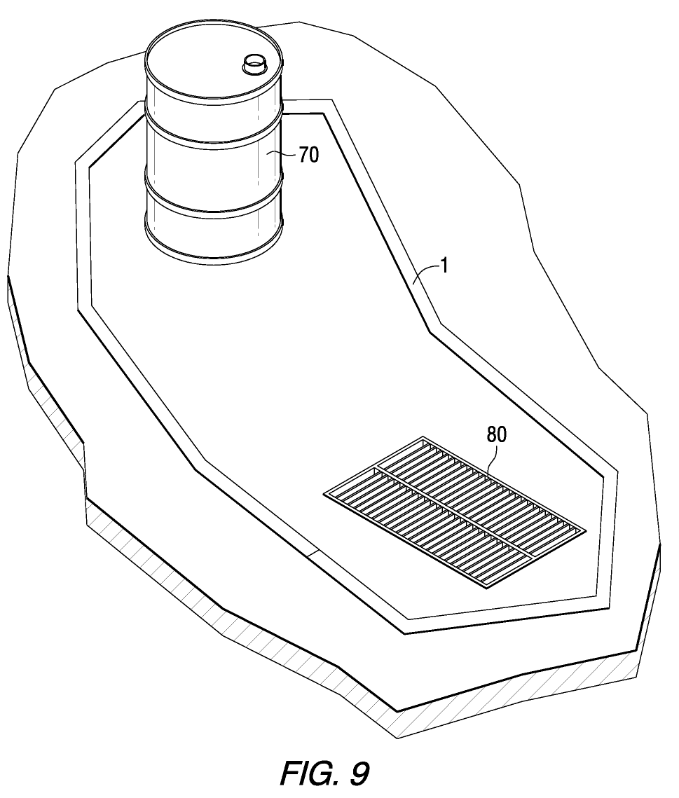

Referring now to FIG. 9, a beneficial aspect of the present invention is illustrated. While it should be apparent that the expanding barrier 1 may provide containment it should also be appreciated that the barrier 1 may also be used as a diverter of liquid. The expanding barrier may be installed around a container 70 or other spill source, such as piping or equipment. Under normal conditions the barrier 1 is low profile and not an obstacle or impediment to pedestrian or wheeled traffic. However, it will be appreciated that should a leak in the container develop, the barrier will expand and divert the liquid to the drain 80 or other receptacle.

Whereas particular embodiments of this invention have been described above for purposes of illustration, it will be evident to those skilled in the art that numerous variations of the details of the present invention may be made without departing from the invention as defined in the appended claims.

1. An expandable liquid barrier comprising:
an adhesive layer attached to a base surface;
an expanding layer attached to the adhesive layer; and
an expandable fill disposed between the adhesive layer and the expanding layer,
wherein liquid contact with the expandable fill causes the expandable fill to swell.

2. The expandable liquid barrier of claim 1 wherein the expandable fill is a super absorbent polymer.

3. The expandable liquid barrier of claim 1 wherein the expandable fill is a compressed sponge.

4. The expandable liquid barrier of claim 1 wherein the expandable fill is foam.

5. The expandable liquid barrier of claim 1 wherein the adhesive layer is a one-sided tape.

6. The expandable liquid barrier of claim 1 wherein a separate adhesive secures the adhesive layer to the base surface.

7. The expandable liquid barrier of claim 1 wherein the barrier has a front longitudinal edge and a rear longitudinal edge, wherein the expanding layer is attached to the adhesive layer along the front longitudinal edge and the rear longitudinal edge.

8. The expandable liquid barrier of claim 1 wherein the barrier has a front longitudinal edge and a rear longitudinal edge, wherein the expanding layer is directly attached to the adhesive layer along the rear longitudinal edge, wherein the expanding layer is secured to the adhesive layer along front longitudinal edge by way of flexible members.

9. An expandable liquid barrier comprising:
an adhesive layer attached to a base surface; and
an expanding layer attached to the adhesive layer comprising a resilient material and soluble adhesive, the expanding layer folded upon itself and restrained in a low profile position by the water-soluble adhesive;
whereby liquid contact with the barrier causes the soluble adhesive to dissolve releasing the resilient expanding layer.

10. The expandable liquid barrier of claim 9 wherein the liquid is water-based and the soluble adhesive is selected from the group consisting of cellulose ether, polyvinylpyrrolidone, and polyvinyl alcohol.

11. The expandable liquid barrier of claim 9 wherein the liquid is oil-based and the soluble adhesive is Ethoxylated nonylphenol or a Transition metal alkyl sulfonate.

12. An expandable liquid barrier comprising:
an adhesive layer attached to a base surface;
a contracting section connected to the adhesive layer and adjacent the base surface; and
an expanding layer attached to the contracting section;
whereby liquid contact with the contracting section causes the contracting section to shrink which bends and increases the profile of the expanding layer.

13. An expandable liquid barrier comprising:
an adhesive layer attached to a base surface;
an expanding layer attached to the adhesive layer; and
means for expanding and heightening the barrier when the barrier is contacted by a liquid.

14. The expandable liquid barrier of claim 13 further comprising a switch in communication with the barrier, whereby the barrier activates a switch when liquid is present.

15. The expandable liquid barrier of claim 13 wherein the expandable liquid barrier provides containment of a liquid.

16. The expandable liquid barrier of claim 13 wherein the expandable liquid barrier diverts the liquid to a receptacle or drain.

17. A method for containing a leaking or spilled liquid comprising the steps of:
providing an expandable, low profile barrier which expands upon contact with the spilled or leaking liquid; and
affixing the expandable, low profile barrier to a base surface.
